(19) United States
(12) Plant Patent Application Publication
(10) Pub. No.: US 2003/0014780 P1
(43) Pub. Date: Jan. 16, 2003

(54) Ground cover rose plant named meirameca

(76) Inventor: Alain A. Meilland, Antibes (FR)

Correspondence Address: BURNS DOANE SWECKER & MATHIS L L P, POST OFFICE BOX 1404, ALEXANDRIA, VA, 22313-1404, US

(21) Appl. No.: 09/698,269

(22) Filed: Jan. 8, 2001

(51) Int. Cl. A01H 5/00

(52) U.S. Cl. PLT/107

(57) ABSTRACT

A new and distinct variety of ground cover rose plant (iv, garden rose plant) is provided which forms in abundance attractive semi-double blossoms that are pink in coloration. The vegetation is vigorous and the growth habit is creeping. Attractive decorative medium green glossy foliage is formed. Excellent disease resistance particularly with respect to Blackspot and Powdery Mildew is exhibited. The new variety grows very well on its own roots following asexual reproduction by cuttings. Attractive ornamentation in the landscape is provided.

### SUMMARY OF THE INVENTION

The new variety of Rosa hybrida Ground Cover rose plant of the present invention was created by artificial pollination wherein two parents were crossed which previously had been studied in the hope that they would contribute the desired characteristics. The female parent (i, the seed parent) of the new variety was the ‘Noatraum’ variety (U.S. Plant Pat. No. 7,282). The male parent (iv, the pollen parent) of the new variety was the product of the cross between the ‘Meibonrib’ variety (U.S. Plant Pat. No. 9,469) and the ‘Korimo’ variety (non-patented in the United States). The ‘Noatraum’ parent variety sometimes is known as the ‘Flower Carpet’ variety. The parentage of the new variety can be summarized as follows:

‘Noatraum’×(‘Meibonrib’×‘Korimo’).

The seeds resulting from the above pollination were sown and small plants were obtained which were physically and biologically different from each other. Selective study resulted in the identification of a single plant of the new variety.

It was found that the new variety of ground cover rose plant of the present invention possesses the following combination of characteristics:

(a) abundantly forms attractive semi-double blossoms that are pink in coloration,

(b) exhibits a creeping growth habit,

(c) forms vigorous vegetation,

(d) forms decorative medium green glossy foliage,

(e) propagates well by cuttings, and

(f) exhibits excellent disease resistance particularly with respect to Blackspot and Powdery Mildew.

The new variety well meets the needs of the horticultural industry. It can be grown to advantage as attractive ornamentation in parks, gardens, public areas, and residential landscapes. Accordingly, it is particularly well suited for growing in the landscape. The pink blossoms contrast nicely with the medium green glossy foliage.

The characteristics of the new variety have been found to be homogeneous and stable and are strictly transmissible by asexual propagation such as budding, grafting, and cuttage from one generation to another. The plant propagates well and grows very well on its own roots when propagated from cuttings.

The new variety has been named the ‘Meirameca’ variety.

### BRIEF DESCRIPTION OF THE PHOTOGRAPHS

The accompanying photograph shows, as nearly true as it is reasonably possible to make the same in a color illustration of this character, typical specimens of the plant and plant parts of the new variety. The rose plants of the new variety illustrated herein were one year of age and were grown outdoors during June on their own roots at LeCannet-des-Nlaures, Var, France. Dimensions in centimeters are indicated at the bottom of the photograph.

FIG. 1—illustrates a specimen of a young shoot;

FIG. 2—illustrates specimens of floral buds before the opening of the sepals;

FIG. 3—illustrates specimens of floral buds at the opening of the sepals;

FIG. 4—illustrates specimens of floral buds at the opening of the petals;

FIG. 5—illustrates specimens of flowers in the course of opening;

FIG. 6—illustrates specimen of an open flower—plan view—obverse;

FIG. 7—illustrates a specimen of an open flower—plan view—reverse;

FIG. 8—illustrates a specimen of a full open flower—plan view—obverse;

FIG. 9—illustrates a specimen of a fully open flower—plan view—reverse;

FIG. 10—illustrates a specimen of a floral receptacle showing the arrangement of the stamens and pistils;

FIG. 11—illustrates a specimen of a floral receptacle showing the arrangement of the pistils (stamens removed);

FIG. 12—illustrates a specimen of a flowering stem;

FIG. 13—illustrates a specimen of a main branch;

FIG. 14—illustrates specimens of leaves with three leaflets—plan view upper surface and under surface;

FIG. 15—illustrates specimens of leaves with five leaflets—plan view upper surface and under surface; and

FIG. 16—illustrates specimens of leaves with seven leaflets—plan view upper surface and under surface.

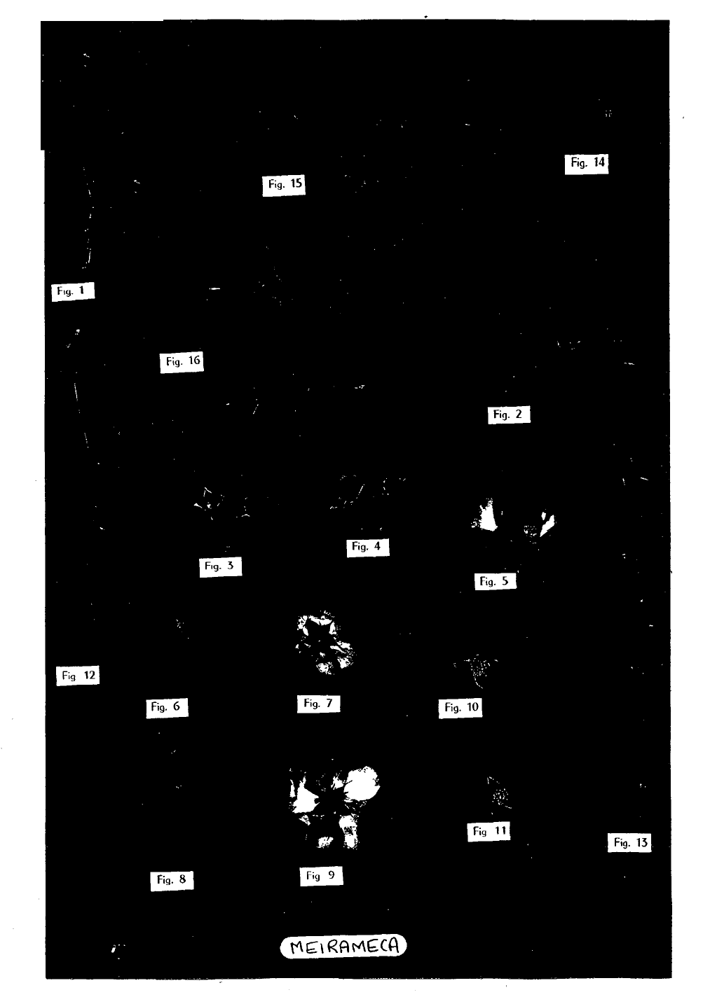

### DETAILED DESCRIPTION

The chart used in the identification of colors is that of The Royal Horticultural Society (R.H.S. Colour Chart). The terminology which precedes reference to the chart has been added to indicate the corresponding color in more common terms. The description is based on one year-old specimens of the new variety during September while grown outdoors on their own roots at LeCannet-des-Maures, Var, France.

Class: Ground Cover (Garden Rose).

Plant:

Height.—approximately 50 to 60 cm. on average at the end of the growing season.

Habit.—creeping ground cover.

Branches:

Color.—young stems very light green suffused with reddish coloration. adult wood: near Yellow-Green Group 146C.

Thorns.—size: medium. quantity: moderately numerous. color: tan on adult wood.

Leaves:

Stipules.—adnate, pectinate, and rather broad.

Petioles.—upper surface: slightly glandular. under surface: with a few prickles.

Leaflets.—number: 3, 5, and 7. shape: oval. serration: regular. texture: consistent. overall appearance: dense, medium green in coloration, and glossy. color (young foliage: upper surface: near Yellow-Green Group 146B. under surface: near Yellow-Green Group 146C. color (adult foliage). upper surface: near Yellow-Green Group 147A. under surface near Yellow-Green Group 147B.

Inflorescence:

Number of flowers.—commonly approximately 6 to 15 and sometimes more blooms per floral stem.

Peduncle.—small, rigid, and approximately 1.5 to 2 cm in length on average. upper surface: greenish in coloration. under surface: green with some reddish coloration.

Bud.—shape: globular. length: approximately 1.5 cm on average. size small. length: approximately 1 cm. color (when opening) upper surface: Spinal Red, Red Group 54B, and suffused with Claret Rose, Red Group 50A. under surface: Red Group 55D and lightly suffused with Claret Rose, Red Group 50B.

Flower.—form: flattened, and semi-double. diameter approximately 5 cm on average. color (when opening begins): upper surface: Spinal Red, near Red Group 54B, and suffused with Claret Rose, Red Group 50A. under surface: near Red Group 55D and lightly suffused with Claret Rose, Red Group 50B. color (when blooming): upper surface: Spinal Red, near Red Group 54A, and suffused with Claret Rose, Red Group 50A. under surface: Spinal Red, near Red Group 54B. color (at end of blooming): upper surface: Spinal Red, near Red Group 54A, and suffused with Claret Rose, Red Group 50A. under surface: Spinal Red, near Red Group 54B. fragrance: none. lasting quality: very long on the plant. stamen number: approximately 85 on average. anthers: yellow-orange in coloration. filamens: yellow in coloration. pistils: approximately 23 on average. stigmas: greenish in coloration. styles: greenish in coloration. receptacle: reddish green in coloration, and in longitudinal section it is in the shape of a pear.

Development:

Vegetation.—very strong.

Blossoming.—very abundant.

Resistance to diseases.—excellent, especially with respect to Blackspot and Powdery Mildew.

I claim:

1. A new and distinct variety of Ground Cover rose plant characterized by the following combination of characteristics:
(a) abundantly forms attractive semi-double blossoms that are pink in coloration,
(b) exhibits a creeping growth habit,
(c) forms vigorous vegetation,
(d) forms decorative medium green glossy foliage,
(e) propagates well by cuttings, and
(f) exhibits excellent disease resistance particularly with respect to Blackspot and Powdery Mildew; substantially as herein shown and described.
